SEQUENCE LISTING (14 sequences)

SEQ ID NO: 1 - DNA, 294 bp, synthetic construct
  source 1..294: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaa

SEQ ID NO: 2 - DNA, 1584 bp, synthetic construct
  source 1..1584: mol_type = other DNA
atggctgatagccgtgacccggcgagcgatcagatgcagcactggaaagaacagcgtgcagcgcagaaagcggatgttctgaccaccggcgcgggtaacccggttggtgataaactgaacgttatcaccgttggcccgcgtggcccgctgctggttcaggatgttgttttcaccgatgaaatggctcacttcgatcgtgaacgtatcccggaacgtgttgttcacgctaaaggtgcaggcgctttcggttacttcgaagttacccacgatattaccaaatactctaaagcgaaagttttcgaacacatcggcaagaaaaccccgatcgcagttcgcttttctaccgttgctggtgaatctggtagcgctgataccgtgcgtgatccgcgtggtttcgcggttaaattctacaccgaagatggtaactgggatctggttggtaacaacactccgattttcttcatccgtgatccgatcctgttcccgtctttcatccattctcagaaacgtaacccgcagacccacctgaaagatccggacatggtttgggacttctggtctctgcgtccggaatctctgcaccaggttagcttcctgttttctgatcgcggtatcccggatggccaccgccatatgaacggctacggtagccacacctttaaactggttaacgctaacggtgaagcggtttactgcaaattccactacaaaaccgatcagggtattaaaaacctgtctgttgaagatgctgcacgtctgagccaggaagatccggattacggcatccgtgacctgtttaacgcgatcgcgaccggcaaatacccgagctggaccttctacatccaggtgatgaccttcaaccaggcagaaaccttcccgttcaacccgttcgatctgaccaaagtttggccgcacaaagactacccgctgattccggttggtaaactggttctgaaccgcaacccggttaactacttcgcagaagttgaacagatcgccttcgatccgtccaacatgccgccgggtatcgaagcaagcccggataaaatgctgcagggccgtctgttcgcatacccggatacccaccgtcatcgtctgggcccgaactacctgcacatcccggttaactgcccgtaccgtgcacgtgttgccaactaccagcgtgacggcccgatgtgcatgcaggataaccagggcggtgcgccgaactactatccgaacagcttcggcgcaccggaacagcagccgagcgcactggaacacagcatccagtacagcggtgaagttcgccgtttcaacaccgctaacgatgataacgttacccaggttcgtgctttctacgttaacgttctgaacgaagaacagcgtaaacgtctgtgcgaaaacatcgctggccacctgaaagatgcacagatcttcattcagaaaaaagcggttaaaaacttcaccgaagttcacccggattacggttcccacatccaggcactgctggataaatataacgcggaaaaaccgaaaaacgcgatccacaccttcgttcagtctggttctcacctggcggcgcgtgaaaaagctaacctgtaa

SEQ ID NO: 3 - DNA, 1482 bp, synthetic construct
  source 1..1482: mol_type = other DNA
atgagttcaaataaactgacaactagctggggcgctccggttggagataatcaaaactcaatgactgccggttctcgcggaccaactttaattcaagatgtacatttactcgaaaaattggcccatttcaaccgagaacgtgttcctgaacgtgttgttcacgccaaaggagcaggcgcacacggatattttgaagtgacaaacgacgtaacaaaatacacgaaagccgctttcctttctgaagtcggcaaacgcacaccgttgttcatccgtttctcaacagttgccggtgaacttggctctgctgacacagttcgcgacccgcgcggatttgctgttaaattttatactgaagaaggaaactacgacatcgtcggcaacaatacgcctgtattctttatccgcgatgcgattaagttccctgatttcatccatacacaaaaaagagatccaaaaacacacctgaaaaaccctacggctgtatgggatttctggtcactttcaccagagtcactgcaccaagtgacaatcctgatgtctgaccgcggaattcctgcgacacttcgccacatgcacggcttcggaagccatacattcaaatggacaaatgccgaaggcgaaggcgtatggattaaatatcactttaaaacagaacaaggcgtgaaaaaccttgatgtcaatacggcagcaaaaattgccggtgaaaaccctgattaccatacagaagaccttttcaacgcaatcgaaaacggtgattatcctgcatggaaactatatgtgcaaatcatgcctttagaagatgcaaatacgtaccgtttcgatccgtttgatgtcacaaaagtttggtctcaaaaagactacccgttaatcgaggtcggacgcatggttctagacagaaatccggaaaactactttgcagaggtagaacaagcgacattttcacctggaaccctcgtgcctggtattgatgtttcaccggataaaatgcttcaaggtcgactttttgcttatcatgatgcacaccgctaccgtgtcggtgcaaaccatcaagcgctgccaatcaaccgcgcacgcaacaaagtaaacaattatcagcgtgatgggcaaatgcgttttgatgataacggcggcggatctgtgtattacgagcctaacagcttcggcggtccaaaagagtcacctgaggataagcaagcagcatatccggtacaaggtatcgctgacagcgtaagctacgatcactacgatcactacactcaagccggcgatctgtatcgtttaatgagtgaagatgaacgtacccgccttgttgaaaatatcgttaatgccatgaagccggtagaaaaagaagaaatcaagctgcgccaaatcgagcacttctacaaagcggatcctgaatacggaaaacgcgtggcagaaggccttggattgccgattaaaaaagattctggcggcagcagccatcaccatcaccatcactaa

SEQ ID NO: 4 - DNA, 2217 bp, synthetic construct
  source 1..2217: mol_type = other DNA
atgagcacgaccgacgatacccataacacgttatccactggaaaatgtcctttccatcagggggggcatgaccgaagcgcaggcgcagggactgccagccgcgactggtggccgaaccagcttcgcgtggatcttttgaatcaacattccaaccgttctaacccgctgggtgaagactttgactaccgcaaagagtttagcaagttagactactccgccctgaaaggggatctcaaggcgctgctgaccgattcacaaccgtggtggcccgctgactggggcagctatgtcggtttgtttattcgcatggcctggcatggcgctggcacctaccgttctattgatggtcgtggcggcgcgggtcgtggtcaacagcgttttgcgccgcttaactcctggccggataacgtcagcctggataaggcgcgtcgtttgttgtggccgattaagcagaaatatggccagaaaatttcctgggccgacctgtttattctggcgggtaacgtggcgctggaaaactccggcttccgtaccttcggtttcggcgccgggcgtgaagatgtctgggaaccggatctggatgtgaactggggcgatgaaaaagcctggttgactcaccgacaccctgaagcgctggcaaaagcgccgctgggcgcgaccgagatgggccttatctacgttaacccggaagggccggatcacagcggcgaaccactttctgccgccgccgctattcgcgctacctttggcaatatggggatgaacgacgaagagaccgtggcgttgatcgctggcgggcataccctcggtaaaacccacggcgcggcagcggcatcccatgtaggggccgatccggaagccgcgccgattgaagcgcagggcttaggttgggccagcagctatggtagtggcgttggcgcggatgctatcacctccgggctggaagtggtctggacgcagacgccgacccagtggagcaactatttcttcgagaacctgttcaaatatgagtgggtacaaacccgtagtccggctggcgctatccagtttgaagcggtagacgcgccggacatcatcccggacccgttcgatccgtcgaaaaaacgtaagccaaccatgctggtcaccgacctgacgctgcgttttgatccggagttcgagaagatttcccgtcgtttccttaacgatccgcaggcctttaatgaagcctttgctcgtgcctggttcaaactgacgcacagagatatgggaccaaaagcgcgttacatcggaccggaagtgccgaaagaagatctgatctggcaggacccgttgccgcaaccgctctatcagccaacgcaggaagacattatcaacctgaaagcggcgatcgctgcatccgggctttctattagcgagatggtttcggttgcctgggcatccgcgtctactttccgcggcggcgataagcgtggcggcgctaacggcgcgcgtctggcattagcgcctcagcgcgactgggatgtcaacgccgttgcggctcgcgttctgccggtattagaagagatccagaaaacgacgaataaagcctcgctggccgatattattgtgctggcgggcgtggtcggtatcgagcaggcggccgctgctgcgggtgtcagcatcagcgtaccttttgcgccgggccgggtggatgcgcgtcaggatcagaccgacatcgagatgttctcgctgcttgaaccgattgctgatggattccgtaactatcgtgcgcgtctggatgtgtcgacgaccgaatcgctgttgattgataaagcgcagcagttaacgttgaccgcgccggaaatgacggtactggttggcgggatgcgtgtgctgggaaccaactttgacggcagccagaacggtgtctttaccgacagaccgggcgtgctcagcactgacttcttcgctaatctgctggatatgcgttacgagtggaagcccaccgacgacgctaatgagctgttcgaaggccgggatcgtctgactggcgaggtaaaatacacggcgacccgcgccgatctggtgtttggttccaactccgtactgcgcgcgctggcggaagtttacgcgtgtagcgatgcgcacgagaagtttgtgaaggacttcgtcgcggcatgggtgaaagtgatgaacctggaccgtttcgatctgcaaggcggcagcagccatcaccatcaccatcaccatcactaa

SEQ ID NO: 5 - DNA, 66 bp, synthetic construct
  source 1..66: mol_type = other DNA
ggcggcagcagccatcaccatcaccatcacagcagcggcctggtgccgcgcggcagccatactagt

SEQ ID NO: 6 - DNA, 72 bp, synthetic construct
  source 1..72: mol_type = other DNA
ggcggcagcagccatcaccatcaccatcacagcggcggcagccatcaccatcaccatcacagcagcggcggc

SEQ ID NO: 7 - DNA, 42 bp, synthetic construct
  source 1..42: mol_type = other DNA
ggcggcagcagccatcaccatcaccatcacagcagcggcggc

SEQ ID NO: 8 - DNA, 2553 bp, synthetic construct
  source 1..2553: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcacagcagcggcggcatgagcacgaccgacgatacccataacacgttatccactggaaaatgtcctttccatcagggggggcatgaccgaagcgcaggcgcagggactgccagccgcgactggtggccgaaccagcttcgcgtggatcttttgaatcaacattccaaccgttctaacccgctgggtgaagactttgactaccgcaaagagtttagcaagttagactactccgccctgaaaggggatctcaaggcgctgctgaccgattcacaaccgtggtggcccgctgactggggcagctatgtcggtttgtttattcgcatggcctggcatggcgctggcacctaccgttctattgatggtcgtggcggcgcgggtcgtggtcaacagcgttttgcgccgcttaactcctggccggataacgtcagcctggataaggcgcgtcgtttgttgtggccgattaagcagaaatatggccagaaaatttcctgggccgacctgtttattctggcgggtaacgtggcgctggaaaactccggcttccgtaccttcggtttcggcgccgggcgtgaagatgtctgggaaccggatctggatgtgaactggggcgatgaaaaagcctggttgactcaccgacaccctgaagcgctggcaaaagcgccgctgggcgcgaccgagatgggccttatctacgttaacccggaagggccggatcacagcggcgaaccactttctgccgccgccgctattcgcgctacctttggcaatatggggatgaacgacgaagagaccgtggcgttgatcgctggcgggcataccctcggtaaaacccacggcgcggcagcggcatcccatgtaggggccgatccggaagccgcgccgattgaagcgcagggcttaggttgggccagcagctatggtagtggcgttggcgcggatgctatcacctccgggctggaagtggtctggacgcagacgccgacccagtggagcaactatttcttcgagaacctgttcaaatatgagtgggtacaaacccgtagtccggctggcgctatccagtttgaagcggtagacgcgccggacatcatcccggacccgttcgatccgtcgaaaaaacgtaagccaaccatgctggtcaccgacctgacgctgcgttttgatccggagttcgagaagatttcccgtcgtttccttaacgatccgcaggcctttaatgaagcctttgctcgtgcctggttcaaactgacgcacagagatatgggaccaaaagcgcgttacatcggaccggaagtgccgaaagaagatctgatctggcaggacccgttgccgcaaccgctctatcagccaacgcaggaagacattatcaacctgaaagcggcgatcgctgcatccgggctttctattagcgagatggtttcggttgcctgggcatccgcgtctactttccgcggcggcgataagcgtggcggcgctaacggcgcgcgtctggcattagcgcctcagcgcgactgggatgtcaacgccgttgcggctcgcgttctgccggtattagaagagatccagaaaacgacgaataaagcctcgctggccgatattattgtgctggcgggcgtggtcggtatcgagcaggcggccgctgctgcgggtgtcagcatcagcgtaccttttgcgccgggccgggtggatgcgcgtcaggatcagaccgacatcgagatgttctcgctgcttgaaccgattgctgatggattccgtaactatcgtgcgcgtctggatgtgtcgacgaccgaatcgctgttgattgataaagcgcagcagttaacgttgaccgcgccggaaatgacggtactggttggcgggatgcgtgtgctgggaaccaactttgacggcagccagaacggtgtctttaccgacagaccgggcgtgctcagcactgacttcttcgctaatctgctggatatgcgttacgagtggaagcccaccgacgacgctaatgagctgttcgaaggccgggatcgtctgactggcgaggtaaaatacacggcgacccgcgccgatctggtgtttggttccaactccgtactgcgcgcgctggcggaagtttacgcgtgtagcgatgcgcacgagaagtttgtgaaggacttcgtcgcggcatgggtgaaagtgatgaacctggaccgtttcgatctgcaaggcggcagcagccatcaccatcaccatcaccatcactaa

SEQ ID NO: 9 - DNA, 957 bp, synthetic construct
  source 1..957: mol_type = other DNA
aggaggtttgatcctatgggaacaatgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 10 - DNA, 58 bp, synthetic construct
  source 1..58: mol_type = other DNA
gatcgatcgagctcaggaggtttgatcctatgggaacaatgcatacatccgagttgct

SEQ ID NO: 11 - DNA, 49 bp, synthetic construct
  source 1..49: mol_type = other DNA
gatcgatcgtcgacttaaacagcctgttcgatctgttcatccagcagtt

SEQ ID NO: 12 - DNA, 49 bp, synthetic construct
  source 1..49: mol_type = other DNA
gatcgatcatcgatttaagacccactttcacatttaagttgtttttcta

SEQ ID NO: 13 - DNA, 45 bp, synthetic construct
  source 1..45: mol_type = other DNA
cataggatcaaacctcctgctagcacttttctctatcactgatag

SEQ ID NO: 14 - DNA, 1107 bp, synthetic construct
  source 1..1107: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtag

(19) United States
(12) Patent Application Publication
(10) Pub. No.: US 2026/0308603 A1
(43) Pub. Date: Oct. 8, 2026

(54) RECOMBINANT EXPRESSION VECTOR FOR CATALASE SECRETION AND ESCHERICHIA COLI STRAIN TRANSFORMED THEREWITH

(71) Applicant: THE INDUSTRY & ACADEMIC COOPERATION IN CHUNGNAM NATIONAL UNIVERSITY (IAC), Daejeon (KR)

(72) Inventors: Hyo Jin LEE, Daejeon (KR); Heung Jin JEON, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 18/998,390

(22) PCT Filed: Jul. 25, 2023
(86) PCT No.: PCT/KR2023/010737

§ 371 Date: Jan. 24, 2025

(30) Foreign Application Priority Data
Jul. 25, 2022 (KR) 10-2022-0092018

(51) Int. Cl. C12N 15/70; A61K 35/74; A61P 35/00; C07K 14/255; C12N 9/08

(52) U.S. Cl. CPC C12N 15/70; A61K 35/74; A61P 35/00; C07K 14/255; C12N 9/0065; C12Y 111/01006; C12N 2830/003

(57) ABSTRACT

Provided are a recombinant expression vector for secretion of CAT protein and an E. coli strain transformed with the vector. Provided are a recombinant expression vector including a flgM gene, a CAT gene, and a flhDC gene, an E. coli strain transformed with the vector, and a pharmaceutical composition for treating cancer, including the E. coli strain as an active ingredient.

### TECHNICAL FIELD

The present invention relates to a recombinant expression vector for secretion of catalase and an Escherichia coli (E. coli) strain transformed with the same. This patent application claims priority to Republic of Korea Patent Application No. 10-2022-0092018, filed with the Korean Intellectual Property Office on Jul. 25, 2022, the disclosure of which is incorporated herein by reference.

### BACKGROUND ART

Cancer is a life-threatening disease in which uncontrolled cell proliferation invades surrounding tissues and destroys normal cells. As susceptibility to cancer increases due to decreased regulatory capacity of the human body caused by aging, cancer has maintained an undisputed first place among all causes of death for 10 consecutive years since 2007 in an aging society, exceeding twice the mortality rate of heart disease which ranks second.

While strengthening immunity is the most effective response system for treating diseases, cancer cells evade the body's immune response as they are not external invaders. Certain viruses or bacteria infect cancer cells more than normal cells, and the infected cancer cell may become targets of immune cells Accordingly, anticancer treatment methods have been proposed in which immune responses against cancer cells are stimulated by deliberately infection with specific viruses or bacteria. For example, facultative anaerobic bacteria such as E. coli and Salmonella have been shown to selectively colonize and grow in tumors, particularly in hypoxic and necrotic regions (Yu et al., 2004; Min et al., 2008; Jiang et al., 2010). Thus, technologies are being developed to deliver therapeutic substances to cancer cells by using such bacteria, and studies are being conducted to introduce therapeutic genes into these viruses or bacteria for delivery to tumor tissues. However, currently, these substances are produced inside bacteria, with only small amounts secreted outside the bacteria, resulting in reduced therapeutic efficacy. Therefore, there is a need for a device that may not only express therapeutic substances inside bacteria but also efficiently deliver them outside the bacteria.

Meanwhile, hypoxia is a common phenomenon in various solid tumors, myocardial infarction, chronic kidney disease, and glioma. Particularly, rapid proliferation of cancer tissues inhibits vascular tissue formation, inducing hypoxia in solid tumors. Such tumor hypoxia may cause resistance to anticancer drugs by increasing the expression of specific genes and may cause cancer recurrence or metastasis. Catalase (CAT) is an enzyme that decomposes hydrogen peroxide into water and oxygen, and is known to contribute to the alleviation of hypoxia by generating oxygen in hypoxic regions.

The inventors completed this invention by producing a recombinant expression vector including a flgM gene, a CAT gene, and a flhDC gene, and an E. coli strain transformed with the recombinant expression vector, thus allowing tumor hypoxia to be improved and alleviated by inducing oxygen generation in tumor regions. Accordingly, this invention may have synergistic effects in cancer treatment through cancer cell death effects, improvement of antibiotic resistance, inhibition of cancer recurrence or metastasis, and combination with other therapeutic approaches (e.g., chemotherapy, nanodrug therapy, radiotherapy, etc.).

The information described in this background section is solely to enhance understanding of the invention's background and may not include information that constitutes prior art known to those skilled in the relevant field.

### DISCLOSURE OF INVENTION

### Technical Problem

One aspect is to provide a recombinant expression vector including a flgM gene, a CAT gene, and a flhDC gene, wherein the flgM gene, the CAT gene, and the flhDC gene are operably linked to an inducible promoter.

Another aspect is to provide an Escherichia coli (E. coli) strain transformed with the recombinant expression vector.

Another aspect is to provide a pharmaceutical composition for treating cancer including the E. coli strain as an active ingredient.

### Solution to Problem

One aspect is to provide a recombinant expression vector including a flgM gene, a CAT gene, and a flhDC gene, wherein the flgM gene, the CAT gene, and the flhDC gene are operably linked to an inducible promoter.

The present invention relates to a recombinant expression vector including a gene encoding a fusion protein of flgM and CAT, wherein the flgM gene and the CAT gene are linked directly or via a linker. Hereinafter, the fusion protein of flgM and CAT is referred to as “flgM-CAT.”

The term “flgM” as used hereon refers to a protein that represses the σ factor responsible for recruiting RNA polymerase required for late flagellar transcription in the type Ill secretion system of bacteria, or a gene encoding such a protein.

The “type III secretion system” refers to a pathogenic substance delivery system developed in gram-negative bacteria. It is a multi-protein complex structure, a syringe-shaped apparatus (Mota L J et al, Ann Med. (2005); 37(4):234-249) that passes through the host cell membrane and directly injects pathogenic active proteins into the cytoplasm. In this type III secretion system, while the hook-basal body, which is the basic structure that can form flagella on the cell membrane, is being formed, the flgM protein remains in the cytoplasm and acts as an anti-σ²⁸ factor that interferes with the transcription of class III genes, such as flagellin subunit FliC or stator protein MotAB. Subsequently, after completion of the hook-basal body structure, a change in substrate specificity occurs simultaneously within the flagellar secretion system, causing flgM to be secreted out of the cell through the central channel of the hook, and subsequently initiating σ²⁸-dependent transcription within the cell. The secretion mechanism of flgM through the flagellar formation process in the type Ill secretion system is shown in FIG. 2.

In an embodiment, the present invention produces a recombinant expression vector containing the flgM gene and the CAT gene, a protein to be secreted, thereby enabling secretion of flgM through the type III secretion system of the strain and secretion of CAT outside the strain. The CAT genetic information may be obtained from known databases such as GenBank of the National Center for Biotechnology Information (NCBI).

The term “flhDC” as used herein refers to a master regulator of flagellin operon genes, which may regulate the expression of the flgM gene. According to an embodiment, the recombinant expression vector has the flhDC gene along with the flgM gene and CAT gene, thereby contributing to enhancing the expression capacity or secretion capacity of flgM-CAT.

The term “gene” as used herein is to be considered in the broadest sense and may encode a structural protein or a regulatory protein. In this regard, the regulatory protein may include a transcription factor, a heat shock protein, or a protein involved in DNA/RNA replication, transcription and/or translation.

The term “genetic construct” as used herein refers to an aggregate, functional unit, or construct each containing the genetic information of a protein of interest, and may be interpreted in the broadest sense. The genetic construct may refer to a DNA insert in which, for example, a flgM gene, a linker gene, and a CAT gene are operably linked, for enhancing the secretion of flgM-CAT from an Escherichia coli (E. coli) strain for the purpose of the present invention.

The term “vector” as used in refers to a DNA construct containing a nucleotide sequence encoding a target polynucleotide operably linked to a suitable regulatory sequence so as to enable expression of the target polynucleotide in a suitable host. The regulatory sequence may include a promoter capable of initiating transcription, an operator sequence for regulating such transcription, a sequence encoding a suitable mRNA ribosome binding site, and a sequence regulating the termination of transcription and translation. After being transformed into a suitable host, the vector may replicate and function independently of the host genome, or may be integrated into the genome itself. The vector may be any one capable of replicating in a host cell. For example, the vector may be a plasmid, cosmid, virus or bacteriophage in natural or recombinant form. The vector may include selection markers for selecting a host cell containing the vector. The selection markers may be used for screening cells transformed with the vector, wherein the markers may confer selectable phenotypes such as drug resistance, nutritional requirements, resistance to cytotoxic agents, or expression of surface proteins. For example, the marker may be at least one selection marker selected from the group consisting of ampicillin, chloramphenicol, neomycin, puromycin, hygromycin, and zeocin. The vector may include genes involved in replication and/or copy number control such as origin of replication and a promoter, and may include restriction enzyme sites, but is not limited thereto. Appropriate restriction enzymes may be positioned at the upstream and downstream of the genetic construct of the present invention to introduce the genetic construct of the present invention into the vector.

The term “polynucleotide” as used herein may be in the form of RNA or DNA, wherein DNA includes cDNA and synthetic DNA. DNA may be single-stranded or double-stranded. If single stranded, it may be the coding strand or the non-coding (antisense) strand, and the coding sequence may encode an identical polypeptide as a result of the degeneracy or redundancy of the genetic code.

The polynucleotide may also include a variant of the polynucleotide described herein, wherein the variant of the polynucleotide may be a naturally occurring allelic variant of the polynucleotide or a non-naturally occurring variant of the polynucleotide. Allelic variants are alternate forms of polynucleotide sequences that may have one or more nucleotide substitutions, deletions, or additions which do not substantially alter the function of the encoded polynucleotide. It is well known in the art that a single amino acid may be encoded by one or more nucleotide codons and that such polynucleotides may be readily modified to produce alternate polynucleotides encoding the same peptide.

According to an embodiment, the recombinant expression vector may contribute to increasing the expression amount or secretion amount of flgM-CAT by including all of the flgM gene, CAT gene, and flhDC gene. For example, the recombinant expression vector may have enhanced expression capacity or secretion capacity of flgM-CAT by additionally including the flhDC gene along with the flgM gene and CAT gene.

The term “inducible promoter” as used herein refers to a promoter capable of switching the operation of a linked gene by an inducer, for example, an ara promoter, a tac promoter, a lac promoter, a lacUV5 promoter, a Ipp promoter, a pLλ promoter, a pRA promoter, a rac5 promoter, an amp promoter, a recA promoter, a SP6 promoter, a trp promoter, a T7 promoter, a pBAD promoter, a Tet promoter, a trc promoter, a pepT promoter, a sulA promoter, a pol 11 (dinA) promoter, a ruv promoter, a uvrA promoter, a uvrB promoter, a uvrD promoter, a umuDC promoter, a lexA promoter, a cea promoter, a caa promoter, a recN promoter, a pagC promoter, a hip promoter, an ansB promoter, or a pflE promoter. In an embodiment, the promoter may be an ara promoter or a Tet promoter.

According to an embodiment, a recombinant expression vector may have increased expression capacity or secretion capacity of the flgM-CAT, as expression of the gene is regulated by the Tet promoter.

The term “operably linked” may refer to nucleotide sequences linked on a single nucleic acid fragment such that one function is affected by the other.

In a specific embodiment, the expression of the flgM gene, the CAT gene, and the flhDC gene may be regulated by the same inducible promoter. In an embodiment, the promoter may be a Tet promoter.

According to an embodiment, the recombinant vector may have enhanced expression capacity or secretion capacity of flgM-CAT, as the expression of the flgM gene, the CAT gene, and the flhDC gene is regulated by the same inducible promoter.

In an embodiment, the recombinant expression vector may include the flgM gene consisting of the polynucleotide sequence of SEQ ID NO: 1, the CAT gene consisting of the polynucleotide sequence of SEQ ID NO: 4, and the flhDC gene consisting of the polynucleotide sequence of SEQ ID NO: 9. In an embodiment, the recombinant expression vector may include a polynucleotide sequence of SEQ ID NO: 8.

In an embodiment, the flgM gene and the CAT gene may be linked via a linker.

The term “linker” as used herein refers to a polypeptide chain used to link a heterologous domain to a protein of interest or a gene encoding the same. The linker peptide may have an amino acid length of 2 to 50, for example, 2 to 40, 2 to 30, 2 to 20, 2 to 15, 2 to 10, 2 to 5, 5 to 50, 5 to 40, 5 to 30, 5 to 20, 5 to 15, or 5 to 10 amino acids, but is not limited thereto. For example, the linker peptide may be one selected from the group consisting of (GₗSₘ)ₙ (where l, m, and n are each ≥2), (GₗSₘ)ₙ-Hₚ-(GₗSₘ)ₙ (where l, m, and n are each ≥1, H≥6), (G₄S)ₐ(EAAAK)_{b}(G₄S)ₐ (where a and b are each an integer from 1 to 4), (G₄S)ₚ(EAAAK)_{q} (where p and q are each an integer from 1 to 4), (EAAAK)ₓ(G₄S)_{y} (where x and y are each an integer from 1 to 4), A(EAAAK)₄ALEA(EAAAK)₄A, (GSSGGS)ᵢ (where i is an integer from 1 to 4), KESGSVSSEQLAQFRSLD, EGKSSGSGSESKST, GSAGSAAGSGEF, (EAAAK) k (where k is an integer from 1 to 5), CRRRRRREAEAC, GGGGGGGG, GGGGGG, AEAAAKEAAAAKA, PAPAP, VSQTSKLTRAETVFPDV, PLGLWA, TRHRQPRGWE, AGNRVRRSVG, RRRRRRRR, GSHHHHHHSG, GGSSHHHHHHSSGG, GGSSHHHHHHSGGSHHHHHHSSGG, and GGSSHHHHHHSSGLVPRGSHTS, but is not limited thereto. In an embodiment, the linker peptide may be GGSSHHHHHHHSSGG.

In an embodiment, the flgM gene and the CAT gene may be linked via a linker consisting of the polynucleotide sequence of SEQ ID NO: 7.

According to an embodiment, the recombinant expression vector may have enhanced expression capacity or secretion capacity of flgM-CAT by linking the flgM gene and the CAT gene via a gene encoding the GGSSHHHHHHHSSGG linker peptide (SEQ ID NO: 7).

Another aspect is to provide an E. coli strain transformed with the recombinant expression vector. Regarding the E. coli strain, the terms and elements already mentioned are as described above.

In an embodiment, the E. coli strain may be an Escherichia coli strain Nissle 1917 (EcN) strain. According to an embodiment, by using an E. coli strain, for example, an EcN strain, as the strain, the expression capacity or secretion capacity of flgM-CAT may be enhanced.

The term “transformation” as used herein refers to introducing a vector containing a polynucleotide encoding a target protein into a host cell so that the protein encoded by the polynucleotide can be expressed in the host cell. For example, the polynucleotide may be introduced into a host cell in the form of an expression cassette, or may be introduced into a host cell in its own form and operably linked to a sequence necessary for expression in the host cell, but is not limited thereto.

The term “attenuation” as used herein refers to artificially weakening the toxicity of a living pathogen by mutating genes involved in the essential metabolism of the pathogen so that it cannot cause disease in the body but only stimulates the immune system to induce immunity. Genes that cause attenuation of bacterial virulence are well known in the art, and the attenuation may be, for example, loss of function of one or more of the genes aroA, aroC, aroD, aroE, asd, rcsB, or galE. In order to prevent reversion to the wild type and further weaken toxicity during in vivo application, the function of two or more of the genes may be lost.

The E. coli strain may have anticancer activity or anticancer adjuvant activity by targeting the tumor site and increasing oxygen production at the tumor site.

In an embodiment, the E. coli strain may be deposited under the accession number KCTC15492BP.

Another aspect provides a pharmaceutical composition for preventing or treating cancer, including the E. coli strain as an active ingredient. Regarding the pharmaceutical composition, the terms and elements already mentioned are as described above.

The term “cancer” as a disease for prevention or treatment by the composition of the present invention refers to a disease caused by cells having aggressive characteristics that divide and grow ignoring normal growth limits, invasive characteristics that infiltrate surrounding tissues, and metastatic characteristics that spread to other parts of the body. For example, the cancer may be a solid cancer, and may be non-restrictively extended to any tumor containing cancer tissue or cancer cells where the cancer targeting ability of the E. coli strain can be exhibited. For example, the type of cancer may be any one selected from the group consisting of head and neck cancer, skin cancer, pancreatic cancer, colorectal cancer, colon cancer, stomach cancer, liver cancer, large intestine cancer, brain cancer, breast cancer, thyroid cancer, bladder cancer, esophagus cancer, uterine cancer, and lung cancer, but is not limited thereto.

In an embodiment, the composition may be an anticancer therapy adjuvant composition. The term “anticancer therapy adjuvant composition” may be used interchangeably with “anticancer therapy adjuvant” or “anticancer adjuvant”, which may refer to an adjuvant used to inhibit or ameliorate anticancer drug resistance or side effects of anticancer drugs. The term “inhibition” may refer to any action that reduces the side effects or resistance of an anticancer drug by administration of an anticancer adjuvant, and the term “amelioration” may refer to any action where side effects or resistance of the anticancer agent is reduced by administration of anticancer adjuvants, or symptoms caused by cancer are ameliorated or beneficially altered due to reduction of side effects or resistance of the anticancer agent.

In an embodiment, the composition may be administered concurrently, separately, or sequentially with an anticancer agent to enhance the anticancer effect of the anticancer agent, for example, the cancer cell growth inhibition effect or the cancer metastasis inhibition effect. The composition may refer to that the effect produced when administered in combination with an anticancer agent is greater than the effect produced when the composition is administered alone. For example, while the composition may not show significant anti-cancer effects when administered alone, it may enhance the anti-cancer effects of the anti-cancer agent when co-administered with the anti-cancer agent. The term “enhancing the anticancer effect of an anticancer agent” may refer to increasing the sensitivity of an anticancer agent and thereby increasing the persistence of the anticancer effect upon repeated administration of the agent.

In an embodiment, the composition may be for enhancing the anticancer effect of an anticancer agent against anticancer drug-resistant cancer. The term “anticancer agent resistant cancer” refers to a state where prevention, amelioration, or treatment of cancer is not effectively achieved in anticancer therapy by administration of anticancer agents, and specifically, may include not only all types of cancers where cell death and cytotoxic mechanisms induced by anticancer agents are not effective due to decreased sensitivity of cancer cells to anticancer agents, but also cancers where anticancer effects were initially shown following administration of anticancer agents but gradually decreased or disappeared with repeated administration of anticancer agents.

The composition of the present invention may be manufactured by a method known in the pharmaceutical field for use as a drug for preventing or treating cancer, and may be used alone or mixed with pharmaceutically acceptable carriers, forming agents, diluents and the like.

The “pharmaceutically acceptable carrier” as used herein may be determined in part by the particular composition being administered, as well as in part by the particular method used to administer the composition. Accordingly, suitable formulations of the compositions are very diverse. For example, the compositions may be formulated for parenteral (i.e., intramuscular, intradermal, or subcutaneous) administration or nasopharyngeal (i.e., intranasal) administration. Typically, parenteral dosage forms include sterile aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers may include water, alcohol/aqueous solutions, emulsions or suspensions, as well as saline and buffered media. Parenteral vehicles may include sodium chloride solution, Ringer's dextrose, dextrose and sodium chloride, lactated Ringer's, or fixed oils. Preservatives and other additives may also be present, for example, as antibacterial agents, antioxidants, chelating agents, and inert gases.

The dosage of the active ingredient according to the present invention may be appropriately selected depending on the degree of absorption of the active ingredient in the body, the form of the preparation, the age, sex and condition of the patient, the degree of symptoms, etc., and the administration may be once a day or divided into several times. The typical dosage may be 0.001 mg/kg day to 10 g/kg day.

Another aspect provides a method of preventing or treating cancer, including administering, to a subject, an anticancer pharmaceutical composition including the E. coli strain as an active ingredient in an amount effective for preventing or treating cancer. In the method of preventing or treating cancer, the terms and elements already mentioned are as described above.

The subject may be a mammal. The mammal may be a human, a dog, a cat, a cow, a goat, or a pig.

The administration may be performed through any general route as long as it can reach the target tissue. For example, it may be administered through routes such as eye drop administration, intraperitoneal administration, intravenous administration, intramuscular administration, subcutaneous administration, intradermal administration, transdermal patch administration, oral administration, intranasal administration, intrapulmonary administration, and rectal administration, and specifically, it may be administered according to the purpose through a route such as eye drop administration.

In this specification, the terms “treatment” or “treating” or “alleviating” or “ameliorating” are used interchangeably. These terms refer to methods of obtaining beneficial or desired results, including but not limited to therapeutic benefits and/or prophylactic benefits. Therapeutic benefits refer to any therapeutically significant amelioration or effect on one or more diseases, conditions or symptoms under treatment. For prophylactic benefits, the composition may be administered to a subject at risk of developing a particular disease, condition or symptom, or to a subject reporting one or more physiological symptoms of a disease, even if the disease, condition or symptom has not yet manifested.

The terms “effective amount” or “therapeutically effective amount” as used herein refer to an amount of an agent sufficient to produce a beneficial or desired result. The therapeutically effective amount may vary depending on one or more of the subject being treated and condition, the weight and age of the subject, the severity of the condition, the method of administration, etc., and this may be readily determined by a person skilled in the art. Further, the term applies to a dosage that is to provide images for detection by any of the imaging methods described herein. Specific dosages may vary depending on one or more of selected specific agents, subsequent administration regimens, whether it is co-administered with other compounds, timing of administration, a tissue being imaged, and body delivery systems carrying the same.

### Advantageous Effects of Invention

According to the recombinant expression vector according to the aspect, it was confirmed that the expression and secretion of flgM-CAT were significantly enhanced in the E. coli strain transformed with the recombinant expression vector.

According to a recombinant expression vector of one aspect, it was confirmed that when a preparation containing an Escherichia coli (E. coli) strain transformed with the recombinant expression vector was administered, anticancer effects and stability were excellent.

### BRIEF DESCRIPTION OF DRAWINGS

FIG. 1 is a diagram schematically showing a process of extracellular secretion of flgM-CAT, using a type III secretion system.

FIG. 2 is a schematic diagram showing the secretion mechanism of flgM through flagella formation in a type III secretion system.

FIG. 3 is a restriction map of a flgM-CAT-flhDC-pBAD33 plasmid.

FIG. 4 shows the results of a western blot confirming the secretion level of flgM-CAT protein from a strain according to Comparative Example 1.

FIG. 5 shows the results of a western blot confirming the secretion level of flgM-CAT protein from a strain according to Comparative Example 2.

FIG. 6 shows the results of a western blot confirming the secretion level of flgM-CAT protein from a strain according to Comparative Example 3.

FIG. 7 shows the results of a western blot confirming the secretion level of flgM-CAT protein from a strain according to Example 1.

FIGS. 8 and 9 show the results of confirming oxygen generation effects of a strain according to an embodiment.

FIG. 10 shows the results of confirming cancer cell death effects of a strain according to an embodiment through LDH assay.

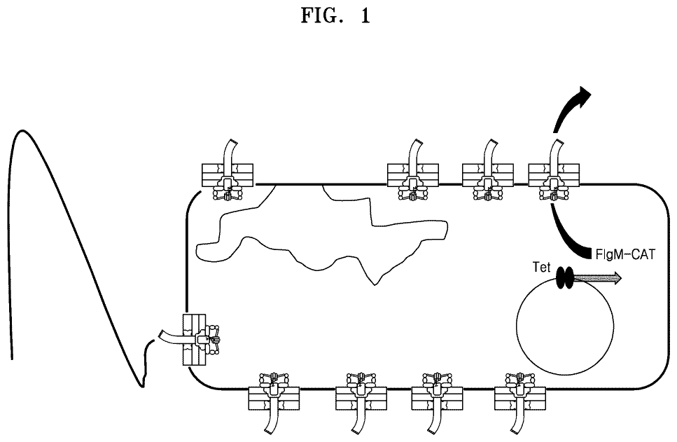

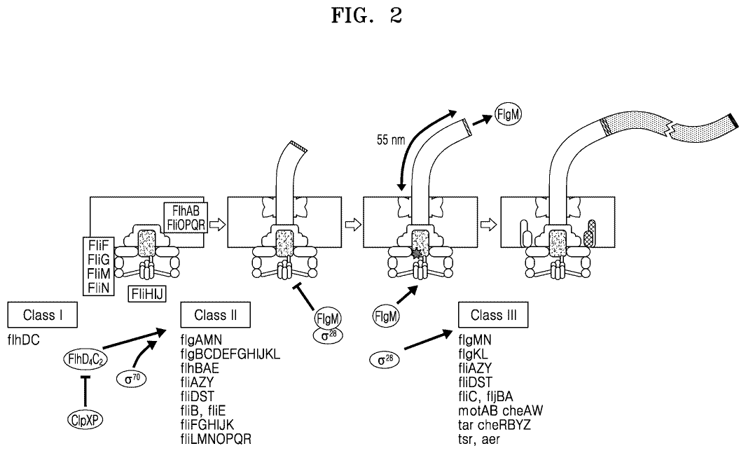

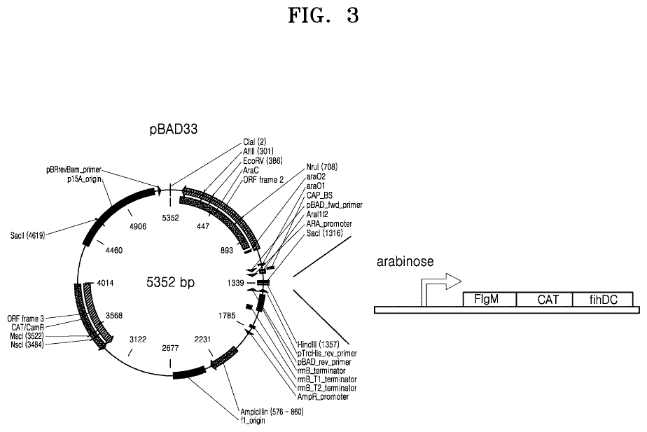

### BEST MODE FOR CARRYING OUT THE INVENTION

Hereinafter, preferred examples are presented to help understand the present invention. However, the following examples are provided only to make it easier to understand the present invention, and the content of the present invention is not limited by the following examples.

### Example 1. Preparation of Plasmids for CAT Secretion and Strains Transformed Therewith

### 1-1. Production of Insert DNA

For preparing a plasmid for CAT secretion, a genetic construct in which the flgM gene and CAT gene are linked to each other was prepared.

Specifically, a genetic construct was prepared by linking, through a linker, a wild-type Salmonella flgM DNA sequence (flgM anti-sigma-28 factor FlgM [Salmonella enterica subsp. enterica serovar Typhimurium str. LT2] Gene ID: 1252690, Locus tagSTM1172 NC_003197.2 (1257036 . . . 1257341, complement)) with a DNA sequence obtained through codon optimization for CAT. To increase the expression of the flgM gene, a shine-dalgarno sequence was inserted in front of the flgM gene. In addition, an nhe I site (GCTAGC) was inserted at the very beginning for cloning, and a Sac I site (GAGCTC) was inserted after the stop codon of CAT. A genetic construct containing flgM, a linker, and CAT was produced through the gene synthesis service of Bionics Co., Ltd., and the genetic construct was treated with 10 U of each of nhe I and Sac I enzymes and reacted at 37° C. for 2 hours. Afterwards, this was subjected to electrophoresis and about 2000 bp DNA was confirmed using a gel extraction kit (Qiagen Co., Ltd.) to obtain flgM-linker-CAT insert DNA.

Thereafter, to obtain flhDC insert DNA, the genomic DNA of wild-type Salmonella LT2 was used as a template and the flhD-Sac1-F and flhC-Sal1-r primers in table 1 were used to obtain a PCR product containing the flhDC gene by PCR. The specific PCR conditions were as follows: 5 μl of 10× buffer, 10 μl of 5×Q solution, 7.5 μl of 2 mM dNTPs, 2.5 μl of 20 μM flhD-Sac1-f primer, 2.5 μl of 20 μM flhC-Sal1-r primer, 1 μl of Taq polymerase, 2 μl of 10 ng/μl genomic DNA, and 19.5 μl of water were mixed, and PCR was performed using the Qiagen system under the following conditions: 95° C. for 5 minutes, 30 cycles of 94° C. for 30 seconds, 49° C. for 30 seconds, and 72° C. for 1 minute, and then 72° C. for 10 minutes. 1 μg of PCR fragment purified using a PCR purification kit was reacted with 10 U of each of Sac I and Sal I at 37° C. for 2 hours, and the reaction product was purified using a PCR purification kit to produce flhDC insert DNA (SEQ ID NO: 9).

TABLE 1

| | | SEQ |
|---|---|---|
| Name | Sequence information | ID NO |
| flhD- | gatcgatcgagctcaggaggtttgatcct | 10 |
| Sac1-f | atgggaacaatgcatacatccgagttgct | |
| flhC- | gatcgatcgtcgacttaaacagcctgttc | 11 |
| Sal1-r | gatctgttcatccagcagtt | |

### 1-2. Preparation of Plasmids

1-2-1. Preparation of flgM-CAT-flhDC-pBAD18-Asd+ Plasmid

Afterwards, using the pBAD18 asd+ plasmid as a vector, 10 U of each of nhe I and Sac I enzymes were reacted at 37° C. for 2 hours, and the reaction products were purified using a PCR purification kit to obtain each vector DNA. The asd gene inserted into the plasmid may consist of the polynucleotide sequence of SEQ ID NO: 14. Afterwards, each vector DNA was ligated with the flgM-CAT insert DNA at 25° C. for 30 minutes and transformed into DH5a competent cells. Thereafter, the transformed cells were plated on LB amp (ampicillin) solid medium and cultured at 37° C. to select colonies having antibiotic resistance. Six candidate colonies were selected from the selected colonies and reacted with 10 U of each of nhe I and Sac I at 37° C. for 1 hour, and formation of about 2000 bp band was confirmed through electrophoresis. Additionally, nucleotide sequence analysis of the plasmid for the candidate colonies was performed to confirm insertion of the flgM, linker, and CAT genes.

1 μg of each plasmid was reacted with 10 U of each of Sac I and Sal I at 37° C. for 2 hours, and the reaction product was purified with PCR purification kit to complete vector DNA. Afterwards, each vector DNA was ligated with the flhDC insert DNA at 25° C. for 30 minutes and then transformed into DH5a competent cells. The transformed cells were plated on LB amp solid medium and cultured at 37° C. to select colonies with antibiotic resistance. Six candidate colonies were selected from the selected colonies and reacted with 10 U of each of Sac I and Sal I at 37° C. for 1 hour, and formation of bands was confirmed through electrophoresis. The insertion of the flhDC gene was confirmed by performing nucleotide sequence analysis on the candidate group.

Through this, flgM-CAT-flhDC-pBAD18-asd+ plasmid was prepared according to Example and Comparative Example.

1-2-2. Preparation of flgM-CAT-flhDC-pBAD33 Plasmid

The flhDC pBAD33 plasmid was provided by Chungnam National University and used as a vector. 10 U of each of nhe I and Sac I enzymes was reacted at 37° C. for 2 hours, and the reaction product was electrophoresed to extract about 6300 bp DNA using Gel extraction kit (Qiagen). Through this, -flhDC-pBAD33 vector was obtained.

Afterwards, the flgM-CAT insert DNA of Example 1-1 and the -flhDC-pBAD33 vector obtained above were ligated at 25° C. for 30 minutes, and then DH5a competent cells were transformed. The transformed cells were plated on LB cm (chloramphenicol) solid medium and cultured at 37° C. for 16 to 24 hours to select colonies resistant to the antibiotic. Six candidate colonies were selected from the selected colonies and reacted with 10 units of Sac I and Sal I at 37° C. for 1 hour, and a band of about 2600 bp was confirmed through electrophoresis, and nucleotide sequence analysis of the candidate colonies was performed. In this way, flgM-CAT-flhDC-pBAD33 plasmid according to an embodiment was prepared.

1-2-3. Preparation of pBAD33 Plasmid with Tet Promoter

The plasmid was prepared as follows to introduce a Tet promoter into the plasmid of Example 1-2-2.

Specifically, to obtain Tet insert DNA, PCR was performed using the strain deposited at the Biological Resource Center of the Korea Research Institute of Bioscience and Biotechnology with the accession number KCTC8808P (Salmonella typhimurium LT2 ΔgalE:tetRA, ΔflhDC::Tn10) (see Korean Patent No. 10-2246740) as a template and the primers in Table 2 below. The specific PCR conditions were as follows: 5 μl of 10× buffer, 10 μl of 5×Q solution, 7.5 μl of 2 mM dNTPs, 2.5 μl of 20 μM TetR-cla1- for primer, 2.5 μl of 20 μM TetR-nhe1-re primer, 1 μl of Taq polymerase, 2 μl of 10 ng/μl genomic DNA, and 19.5 μl of water were mixed, and PCR was performed using the Qiagen system under the following conditions: 95° C. for 5 minutes, 30 cycles of 94° C. for 30 seconds, 49° C. for 30 seconds, and 72° C. for 1 minute, and then 72° C. for 10 minutes. 1 μg of PCR fragment purified with PCR purification kit and 10 U of each of cla1 and nhe1 were reacted at 37° C. for 2 hours, and the reaction product was purified with a PCR purification kit to obtain Tet insert DNA.

TABLE 2

| | | SEQ |
|---|---|---|
| Name | Sequence information | ID NO |
| TetR- | gatcgatcatcgatttaagacccac | 12 |
| cla1-for | tttcacatttaagttgtttttcta | |
| TetR- | CATAGGATCAAACCTCCTGCTAGCA | 13 |
| nhe1-re | CTTTTCTCTATCACTGATAG | |

Subsequently, the flgM-CAT-flhDC-pBAD33 plasmid of Example 1-2-2 was treated with 10 U of each of cla I and nhe I, and reacted at 37° C. for 2 hours. Thereafter, the reaction product was subjected to electrophoresis, and about 7,500 bp of DNA was extracted from the gel using a gel extraction kit (Qiagen), thereby producing the -flgM-CAT-flhDC-pBAD33 vector. The Tet insert DNA and -flgM-CAT-flhDC-pBAD33 vector were ligated at 25° C. for 30 minutes, and transformed into DH5a competent cells. Thereafter, the transformed cells were plated on LB cm solid medium and cultured at 37° C. to select colonies with antibiotic resistance. Six candidate colonies were selected from the selected colonies and treated with 10 units each of cla I and nhe I and reacted at 37° C. for 1 hour, and a band of about 600 bp was confirmed through electrophoresis, and sequence analysis of the candidate colonies was performed. In this way, the Tet-flgM-CAT-flhDC-pBAD33 plasmid was prepared according to an embodiment.

Meanwhile, the genetic information of major constructs in the present embodiment is shown in table 3 below.

TABLE 3

| | | SEQ |
|---|---|---|
| Name | Sequence information | ID NO |
| flgM | atgagcattgaccgtacctcacctttgaaacccgttagcactgtccagacgcgcgaaacc | 1 |
| | agcgacacgccggtacaaaaaacgcgtcaggaaaaaacgtccgccgcgacgagcg | |
| | ccagcgtaacgttaagcgacgcgcaagcgaagctcatgcagccaggcgtcagcgaca | |
| | ttaatatggaacgcgtcgaagcattaaaaacggctatccgtaacggtgagttaaaaatgg | |
| | atacgggaaaaatagcagactcgctcattcgcgaggcgcagagctacttacagagtaa | |
| | aaaa | |
| CAT1 | ATGGCTGATAGCCGTGACCCGGCGAGCGATCAGATGCAGC | 2 |
| | ACTGGAAAGAACAGCGTGCAGCGCAGAAAGCGGATGTTCTG | |
| | ACCACCGGCGCGGGTAACCCGGTTGGTGATAAACTGAACGT | |
| | TATCACCGTTGGCCCGCGTGGCCCGCTGCTGGTTCAGGATG | |
| | TTGTTTTCACCGATGAAATGGCTCACTTCGATCGTGAACGTA | |
| | TCCCGGAACGTGTTGTTCACGCTAAAGGTGCAGGCGCTTTC | |
| | GGTTACTTCGAAGTTACCCACGATATTACCAAATACTCTAAA | |
| | GCGAAAGTTTTCGAACACATCGGCAAGAAAACCCCGATCGC | |
| | AGTTCGCTTTTCTACCGTTGCTGGTGAATCTGGTAGCGCTGA | |
| | TACCGTGCGTGATCCGCGTGGTTTCGCGGTTAAATTCTACA | |
| | CCGAAGATGGTAACTGGGATCTGGTTGGTAACAACACTCCG | |
| | ATTTTCTTCATCCGTGATCCGATCCTGTTCCCGTCTTTCATCC | |
| | ATTCTCAGAAACGTAACCCGCAGACCCACCTGAAAGATCCG | |
| | GACATGGTTTGGGACTTCTGGTCTCTGCGTCCGGAATCTCT | |
| | GCACCAGGTTAGCTTCCTGTTTTCTGATCGCGGTATCCCGG | |
| | ATGGCCACCGCCATATGAACGGCTACGGTAGCCACACCTTT | |
| | AAACTGGTTAACGCTAACGGTGAAGCGGTTTACTGCAAATTC | |
| | CACTACAAAACCGATCAGGGTATTAAAAACCTGTCTGTTGAA | |
| | GATGCTGCACGTCTGAGCCAGGAAGATCCGGATTACGGCAT | |
| | CCGTGACCTGTTTAACGCGATCGCGACCGGCAAATACCCGA | |
| | GCTGGACCTTCTACATCCAGGTGATGACCTTCAACCAGGCA | |
| | GAAACCTTCCCGTTCAACCCGTTCGATCTGACCAAAGTTTGG | |
| | CCGCACAAAGACTACCCGCTGATTCCGGTTGGTAAACTGGT | |
| | TCTGAACCGCAACCCGGTTAACTACTTCGCAGAAGTTGAACA | |
| | GATCGCCTTCGATCCGTCCAACATGCCGCCGGGTATCGAAG | |
| | CAAGCCCGGATAAAATGCTGCAGGGCCGTCTGTTCGCATAC | |
| | CCGGATACCCACCGTCATCGTCTGGGCCCGAACTACCTGCA | |
| | CATCCCGGTTAACTGCCCGTACCGTGCACGTGTTGCCAACT | |
| | ACCAGCGTGACGGCCCGATGTGCATGCAGGATAACCAGGG | |
| | CGGTGCGCCGAACTACTATCCGAACAGCTTCGGCGCACCG | |
| | GAACAGCAGCCGAGCGCACTGGAACACAGCATCCAGTACA | |
| | GCGGTGAAGTTCGCCGTTTCAACACCGCTAACGATGATAAC | |
| | GTTACCCAGGTTCGTGCTTTCTACGTTAACGTTCTGAACGAA | |
| | GAACAGCGTAAACGTCTGTGCGAAAACATCGCTGGCCACCT | |
| | GAAAGATGCACAGATCTTCATTCAGAAAAAAGCGGTTAAAAA | |
| | CTTCACCGAAGTTCACCCGGATTACGGTTCCCACATCCAGG | |
| | CACTGCTGGATAAATATAACGCGGAAAAACCGAAAAACGCG | |
| | ATCCACACCTTCGTTCAGTCTGGTTCTCACCTGGCGGCGCG | |
| | TGAAAAAGCTAACCTGTAA | |
| CAT2 | atgagttcaaataaactgacaactagctggggcgctccggttggagataatcaaaactc | 3 |
| | aatgactgccggttctcgcggaccaactttaattcaagatgtacatttactcgaaaaattgg | |
| | cccatttcaaccgagaacgtgttcctgaacgtgttgttcacgccaaaggagcaggcgcac | |
| | acggatattttgaagtgacaaacgacgtaacaaaatacacgaaagccgctttcctttctga | |
| | agtcggcaaacgcacaccgttgttcatccgtttctcaacagttgccggtgaacttggctctg | |
| | ctgacacagttcgcgacccgcgcggatttgctgttaaattttatactgaagaaggaaacta | |
| | cgacatcgtcggcaacaatacgcctgtattctttatccgcgatgcgattaagttccctgatttc | |
| | atccatacacaaaaaagagatccaaaaacacacctgaaaaaccctacggctgtatgg | |
| | gatttctggtcactttcaccagagtcactgcaccaagtgacaatcctgatgtctgaccgcgg | |
| | aattcctgcgacacttcgccacatgcacggcttcggaagccatacattcaaatggacaaa | |
| | tgccgaaggcgaaggcgtatggattaaatatcactttaaaacagaacaaggcgtgaaa | |
| | aaccttgatgtcaatacggcagcaaaaattgccggtgaaaaccctgattaccatacaga | |
| | agaccttttcaacgcaatcgaaaacggtgattatcctgcatggaaactatatgtgcaaatc | |
| | atgcctttagaagatgcaaatacgtaccgtttcgatccgtttgatgtcacaaaagtttggtctc | |
| | aaaaagactacccgttaatcgaggtcggacgcatggttctagacagaaatccggaaaa | |
| | ctactttgcagaggtagaacaagcgacattttcacctggaaccctcgtgcctggtattgatg | |
| | tttcaccggataaaatgcttcaaggtcgactttttgcttatcatgatgcacaccgctaccgtgt | |
| | cggtgcaaaccatcaagcgctgccaatcaaccgcgcacgcaacaaagtaaacaattat | |
| | cagcgtgatgggcaaatgcgttttgatgataacggcggcggatctgtgtattacgagccta | |
| | acagcttcggcggtccaaaagagtcacctgaggataagcaagcagcatatccggtaca | |
| | aggtatcgctgacagcgtaagctacgatcactacgatcactacactcaagccggcgatct | |
| | gtatcgtttaatgagtgaagatgaacgtacccgccttgttgaaaatatcgttaatgccatga | |
| | agccggtagaaaaagaagaaatcaagctgcgccaaatcgagcacttctacaaagcgg | |
| | atcctgaatacggaaaacgcgtggcagaaggccttggattgccgattaaaaaagattct | |
| | GGCGGCAGCAGCCATCACCATCACCATCACtaa | |
| CAT3 | atgagcacgaccgacgatacccataacacgttatccactggaaaatgtcctttccatcag | 4 |
| | ggggggcatgaccgaagcgcaggcgcagggactgccagccgcgactggtggccga | |
| | accagcttcgcgtggatcttttgaatcaacattccaaccgttctaacccgctgggtgaagac | |
| | tttgactaccgcaaagagtttagcaagttagactactccgccctgaaaggggatctcaag | |
| | gcgctgctgaccgattcacaaccgtggtggcccgctgactggggcagctatgtcggtttgt | |
| | ttattcgcatggcctggcatggcgctggcacctaccgttctattgatggtcgtggcggcgcg | |
| | ggtcgtggtcaacagcgttttgcgccgcttaactcctggccggataacgtcagcctggata | |
| | aggcgcgtcgtttgttgtggccgattaagcagaaatatggccagaaaatttcctgggccg | |
| | acctgtttattctggcgggtaacgtggcgctggaaaactccggcttccgtaccttcggtttcg | |
| | gcgccgggcgtgaagatgtctgggaaccggatctggatgtgaactggggcgatgaaaa | |
| | agcctggttgactcaccgacaccctgaagcgctggcaaaagcgccgctgggcgcgac | |
| | cgagatgggccttatctacgttaacccggaagggccggatcacagcggcgaaccacttt | |
| | ctgccgccgccgctattcgcgctacctttggcaatatggggatgaacgacgaagagacc | |
| | gtggcgttgatcgctggcgggcataccctcggtaaaacccacggcgcggcagcggcat | |
| | cccatgtaggggccgatccggaagccgcgccgattgaagcgcagggcttaggttgggc | |
| | cagcagctatggtagtggcgttggcgcggatgctatcacctccgggctggaagtggtctg | |
| | gacgcagacgccgacccagtggagcaactatttcttcgagaacctgttcaaatatgagtg | |
| | ggtacaaacccgtagtccggctggcgctatccagtttgaagcggtagacgcgccggac | |
| | atcatcccggacccgttcgatccgtcgaaaaaacgtaagccaaccatgctggtcaccga | |
| | cctgacgctgcgttttgatccggagttcgagaagatttcccgtcgtttccttaacgatccgca | |
| | ggcctttaatgaagcctttgctcgtgcctggttcaaactgacgcacagagatatgggacca | |
| | aaagcgcgttacatcggaccggaagtgccgaaagaagatctgatctggcaggacccgt | |
| | tgccgcaaccgctctatcagccaacgcaggaagacattatcaacctgaaagcggcgat | |
| | cgctgcatccgggctttctattagcgagatggtttcggttgcctgggcatccgcgtctactttc | |
| | cgcggcggcgataagcgtggcggcgctaacggcgcgcgtctggcattagcgcctcagc | |
| | gcgactgggatgtcaacgccgttgcggctcgcgttctgccggtattagaagagatccaga | |
| | aaacgacgaataaagcctcgctggccgatattattgtgctggcgggcgtggtcggtatcg | |
| | agcaggcggccgctgctgcgggtgtcagcatcagcgtaccttttgcgccgggccgggtg | |
| | gatgcgcgtcaggatcagaccgacatcgagatgttctcgctgcttgaaccgattgctgatg | |
| | gattccgtaactatcgtgcgcgtctggatgtgtcgacgaccgaatcgctgttgattgataaa | |
| | gcgcagcagttaacgttgaccgcgccggaaatgacggtactggttggcgggatgcgtgt | |
| | gctgggaaccaactttgacggcagccagaacggtgtctttaccgacagaccgggcgtg | |
| | ctcagcactgacttcttcgctaatctgctggatatgcgttacgagtggaagcccaccgacg | |
| | acgctaatgagctgttcgaaggccgggatcgtctgactggcgaggtaaaatacacggcg | |
| | acccgcgccgatctggtgtttggttccaactccgtactgcgcgcgctggcggaagtttacg | |
| | cgtgtagcgatgcgcacgagaagtttgtgaaggacttcgtcgcggcatgggtgaaagtg | |
| | atgaacctggaccgtttcgatctgcaaGGCGGCAGCAGCCATCACCATC | |
| | ACCATCACCATCACTAA | |
| linker 1 | GGCGGCAGCAGCCATCACCATCACCATCACAGCAGCGGCC | 5 |
| | TGGTGCCGCGCGGCAGCCATACTAGT | |
| linker 2 | GGCGGCAGCAGCCATCACCATCACCATCACAGCGGCGGCA | 6 |
| | GCCATCACCATCACCATCACAGCAGCGGCGGC | |
| linker 3 | GGCGGCAGCAGCCATCACCATCACCATCACAGCAGCGGCG | 7 |
| | GC | |
| flhDC | AGGAGGTTTGATCCTatgggaacaatgcatacatccgagttgctaaaacacat | 9 |
| | ttatgacatcaatttgtcatatttactccttgcacagcgtttgatcgtccaggacaaagcatct | |
| | gcgatgttccgcctcggtatcaacgaagagatggcaaacacactgggcgcgttgaccct | |
| | gccgcagatggtcaaactggcggagacgaaccagttagtttgtcatttccggtttgacgat | |
| | catcagacgatcacccgtttgactcaggattcgcgcgtcgatgacttacagcagattcaca | |
| | caggtatcatgctttcaacgcgtctgctcaatgaagtggacgatacggcgcgtaagaaaa | |
| | gggcatgaaagggcatgataatgagtgaaaaaagcattgttcaggaagctcgcgatatc | |
| | cagttggcgatggagttgattaatcttggcgctcgtctacaaatgctggaaagcgaaaca | |
| | cagctcagccgtggtcgcctcatcaggctgtacaaagaattacgcggtagcccgccgcc | |
| | taaagggatgctgccattttcgacagactggtttatgacctgggagcaaaatattcatgcct | |
| | ccatgttctgcaacgcctggcaatttttactgaagaccggcttatgcagcggtgtggatgcg | |
| | gtgattaaagcttatcggctttatcttgagcagtgtccgcaaccgcctgaagggccgttgttg | |
| | gcgctgactcgcgcatggacgctggtgcgttttgttgaaagtgggttgcttgaattgtcgag | |
| | ctgtaactgctgcggtgggaactttattacccatgcgcatcagcccgtaggcagctttgcgt | |
| | gtagtttatgccagccgccatcccgcgcagtaaaaagacgtaaactttcccgagatgctg | |
| | ccgatattattccacaactgctggatgaacagatcgaacaggctgtttaa | |

1-3. Production of Escherichia coli (E. coli) Strain Secreting flgM-CAT

The plasmids of Examples 1-2 were each transformed into the following strains: BRD 509, BRD509 asd rcsB galE: tetRA-(#1323), and EcN (E. coli Nissle 1917) strains. The BRD 509 and BRD509 asd rcsB galE: tetRA-(#1323) strains were provided by Chungnam National University, and the EcN strain was provided by Chonnam National University. The genetic and strain information of the transformed strain are as shown in table 4 below.

TABLE 4

| | | Comparative | Comparative | Comparative |
|---|---|---|---|---|
| | Example 1 | Example 1 | Example 2 | Example 3 |
| CAT-linker | CAT3- | CAT1- | Cat2- | Cat3- |
| gene | linker 3 | linker 1 | linker 2 | linker 3 |
| Plasmid | pBAD33 | pBAD18 asd+ | pBAD18 asd+ | pBAD33 |
| Strain | EcN | #1323 | #1323 | BRD 509 |
| Promoter | Tet | ara | ara | ara |

The present inventors deposited the flgM-CAT-flhDC-pBAD33/EcN strain according to an embodiment of the present invention to the Biological Resource Center of the Korea Research Institute of Bioscience and Biotechnology on Jun. 29, 2023, and were assigned the accession number KCTC15492BP (flgM-CAT4-flhDC-pBAD33/EcN).

### Experimental Example 1. Confirmation of Protein Expression and Secretion Capacity

Western blot analysis was performed to confirm flgM-CAT expression and secretion capacities of an E. coli strain transformed with the recombinant expression vector according to an embodiment by the following method.

Specifically, a single colony of each transformed strain was inoculated in LB amp liquid medium (Comparative Examples 1 and 2) or LB cm liquid medium (Comparative Example 3 and Example 1) and shake-cultured at 37° C. for 12 to 16 hours. Subsequently, the culture was diluted in fresh LB amp liquid medium or LB cm liquid medium to OD600 of 0.05 and shake-cultured at 37° C. for 2 hours. When OD600 reached 0.4 to 0.6, groups treated with arabinose (Comparative Examples 1 to 3, final concentration 0.2% (w/v)) or doxycycline (Example 1, final concentration 100 ng/ml) were designated as test groups (+), and untreated groups as control groups (−), and shake-cultured additionally at 37° C. for 5 hours. Subsequently, samples from the test and control groups were separated into a whole cell culture, pellets, and a supernatant. The pellets and the supernatant were separated by centrifuging the whole cell culture at 8000 rpm. The supernatant was further filtered through a 0.2 μm filter to completely remove bacteria and concentrated using nanosep (OD010C35 3K omega) from Pall company.

50 μl of each of whole cell culture, pellets and the supernatant samples were mixed with SDS sample buffer to prepare samples for western blot. Each sample was denatured in a 100° C. heating block for 5 minutes, centrifuged at 13,000 rpm and 4° C. for 5 minutes, and electrophoresed on a 10% polyacrylamide gel. The gel was transferred using PVDF (polyvinylidene fluoride) to move the proteins onto a PVDF membrane, and the membrane was blocked using a blocking buffer at room temperature for 1 hour. Afterwards, anti-his tag (SB194b, Southern Biotech) was diluted 1/1000 as the primary antibody and reacted at 4° C. for 16 hours. Afterwards, the membrane was washed three times with TBST (Tris-buffered saline with 0.1% Tween-20) at 10-minute intervals, and then reacted with mouse anti-mouse-IgG (#7076s, Cell signaling, Danvers, MA, USA) secondary antibody at room temperature for 1 hour. After reaction, the membrane was washed three times with TBST at 10-minute intervals, exposed to X-ray film using ECL buffer, and developed to confirm protein expression levels.

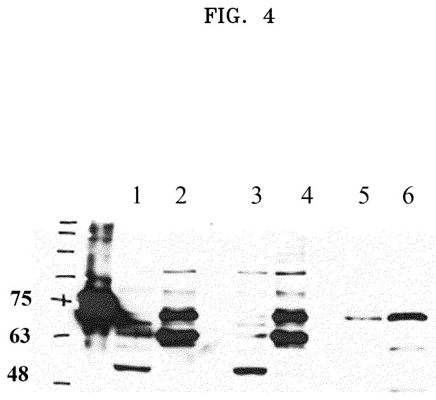

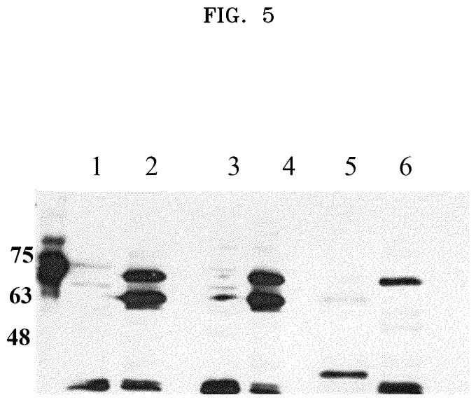

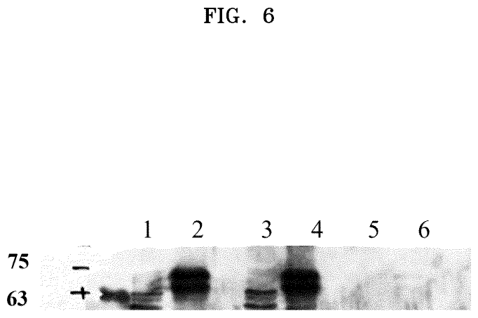

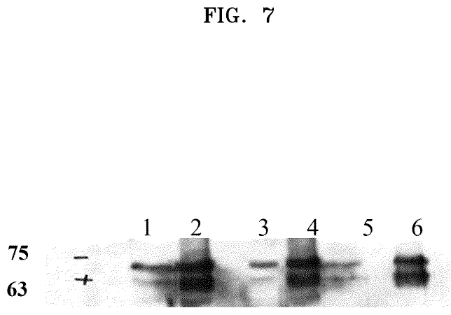

FIG. 4 shows results from the strain according to Comparative Example 1, FIG. 5 shows results from the strain according to Comparative Example 2, FIG. 6 shows results from the strain according to Comparative Example 3, and FIG. 7 shows results from the strain according to Example 1. Lanes 2, 4, and 6 of FIGS. 4 to 7 are test groups treated with arabinose or doxycycline, and lanes 1, 3, and 5 are control groups not treated with arabinose or doxycycline. Lanes 1 and 2 of FIGS. 4 to 7 show results for whole cell (W.C), lanes 3 and 4 show results for pellets, and lanes 5 and 6 show results for the supernatant. Lanes 5 and 6 of FIGS. 4 and 5 show results obtained by concentrating 30 ml of the supernatant.

As a result, as shown in FIG. 7, for the strain according to an embodiment, secretion of flgM-CAT protein was confirmed even in the bacteria-removed supernatant. Additionally, the protein was detected at high levels even in low concentration bacterial culture, confirming excellent flgM-CAT expression and secretion capacity of the strain according to an embodiment. Through this, it was confirmed that the strain according to an embodiment not only expressed flgM-CAT at high levels but also secreted the protein at high levels outside the strain.

### Experimental Example 2. Confirmation of the Oxygen-Generating Effect of the Strain

To confirm the oxygen-generating effect of the strain according to an embodiment, the following experiment was performed.

### 2-1. Preparation of Strain Culture Solution

A single colony of the strain according to an embodiment was inoculated into LB cm liquid medium and cultured in a shaking incubator at 37° C. for 12 to 16 hours. Afterwards, the cells were adjusted to OD600 of 0.05 in fresh LB cm liquid medium and cultured with shaking at 37° C. for 2 hours. When OD600 reached 0.4 to 0.6, groups treated with doxycycline (final concentration 100 ng/ml) were designated as test groups (+), and untreated groups as control groups (−), and shake-cultured additionally at 37° C. for 5 hours. Subsequently, the culture medium was centrifuged at 3000 rpm to separate the supernatant, and the supernatant was further filtered through a 0.2 μm filter to completely remove bacteria and concentrated 30-fold using nanosep (OD010C35 3K omega) from Pall company.

### 2-2. Confirmation of Oxygen Generation Effect of Strain Culture Solution

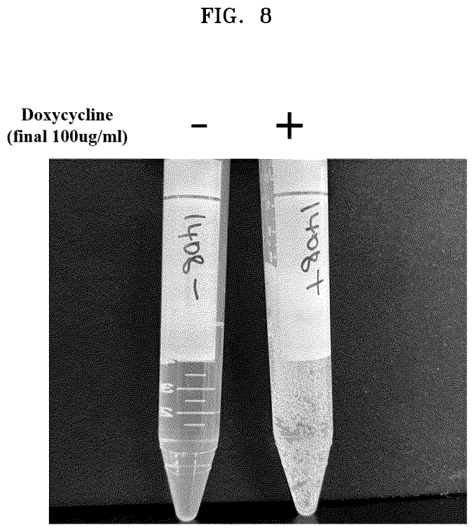

50 μl of the concentrated strain culture of Experimental Example 2-1 above was added to 5 ml of 3% H₂O₂ and oxygen generation was observed. As a result, as shown in FIG. 8, it was confirmed that bubble generation was significantly higher in the doxycycline-treated group. Through this, it was confirmed that oxygen generation was significantly increased when flgM-CAT expression was induced by doxycycline treatment.

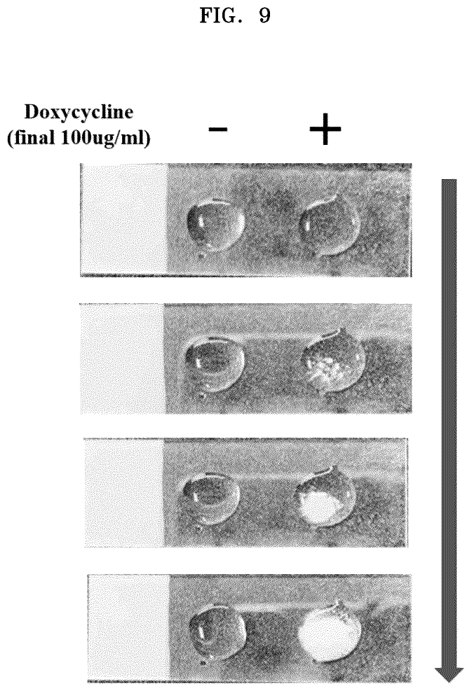

Additionally, 200 μl of 3% H₂O₂ was placed on a slide glass, treated with 20 μl of the concentrated strain culture medium from Experimental Example 2-1, and bubble generation was observed over time. As a result, as shown in FIG. 9, it was confirmed that bubble generation increased significantly over time. Through this, it was confirmed that oxygen generation was significantly increased as flgM-CAT expression was induced by doxycycline treatment. In this way, it was confirmed that the strain according to an embodiment may significantly increase oxygen production by expressing and secreting flgM-CAT at high levels.

### Experimental Example 3. Confirmation of Anticancer Effect

Experiments were performed as follows to confirm cancer cell death effects of a strain according to an embodiment.

### 3-1. Tumor Cell Line Culture

Human colon cancer cell line SW480 was purchased from the American Type Culture Collection. The cells were cultured in Dulbecco's modified Eagle's medium (DMEM) containing 10% fetal bovine serum (FBS) and 1% penicillin-streptomycin. When the cells were 2×10⁵, the cells were seeded in a 6-well plate. 24 hours after seeding, 50 μl of the supernatant (flgM-CAT) obtained in Experimental Example 2-1 was treated and cultured for 48 hours.

### 3-2. Confirmation of Tumor Cell Killing Effect

To measure the extent of cell death of tumor cells by the strain according to an embodiment, an LDH assay was performed using DogenBio's EZ-LDH (DG-LDH500) assay kit as follows. Specifically, the culture medium of Experimental Example 3-1 was centrifuged at 1000 rpm for 3 minutes to remove cell debris, and then 100 μl of LDH solution was mixed with 10 μl of the supernatant treated in this manner and incubated at room temperature for 30 minutes. Afterwards, LDH release was measured by measuring absorbance using a 450 nm microreader. All experiments were repeated three times.

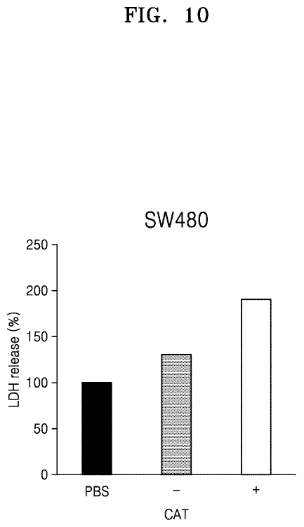

As a result, as shown in FIG. 10, it was confirmed that LDH release was significantly higher in the doxycycline-treated group (+) than in the doxycycline-untreated group (−). Through this, the excellent cancer cell killing effect of the strain according to an embodiment was confirmed. In addition, since it was confirmed that such cell death was due to cell membrane damage, it was also confirmed that this could show superior anticancer effects by inducing secondary anticancer immune effects through stimulation of immune cells by intracellular components.

In this way, it was confirmed that the strain according to an embodiment may reduce or ameliorate hypoxia by generating oxygen at the tumor site by secreting flgM-CAT at the tumor site. Additionally, cancer cell death effects of the strain according to an embodiment were confirmed. Through this, it was confirmed that the strain may increase sensitivity to radiation therapy, decrease the expression of HIF-1α, decrease or improve resistance to various anticancer drugs, and exhibit the effect of inhibiting tumor recurrence and metastasis by killing cancer cells and reducing hypoxia in the tumor area.

While the specific aspects of the present invention have been described in detail above, it would be apparent to those skilled in the art that such specific description is only a preferred embodiment, and the scope of the present invention is not limited thereby. Accordingly, the substantial scope of the present invention will be defined by the appended claims and their equivalents.

### ACCESSION NUMBER

Depository Authority: Korean Collection for Type Cultures (KCTC), Korea Research Institute of Bioscience and Biotechnology
Accession number: KCTC15492BP
Date of Deposit: 20230629

1. A recombinant expression vector comprising a flgM gene, a CAT gene, and a flhDC gene, wherein the flgM gene, the CAT gene, and the flhDC gene are operably linked to an inducible promoter.

2. The recombinant expression vector of claim 1, wherein expression of the flgM gene, the CAT gene, and the flhDC gene is regulated by the same inducible promoter.

3. The recombinant expression vector of claim 1, wherein the promoter is a Tet promoter.

4. The recombinant expression vector of claim 1, wherein the recombinant expression vector comprises the flgM gene consisting of a polynucleotide sequence of SEQ ID NO: 1, the CAT gene consisting of a polynucleotide sequence of SEQ ID NO: 4, and the flhDC gene consisting of a polynucleotide sequence of SEQ ID NO: 9.

5. The recombinant expression vector of claim 1, wherein the flgM gene and the CAT gene are linked to each other via a linker.

6. The recombinant expression vector of claim 5, wherein the linker consists of a polynucleotide sequence of SEQ ID NO: 7.

7. An E. coli strain transformed with the recombinant expression vector of claim 1.

8. The E. coli strain of claim 7, wherein the E. coli strain has enhanced capacity for secretion of CAT protein.

9. The E. coli strain of claim 7, wherein the E. coli strain is deposited under accession number KCTC15492BP.

10. A pharmaceutical composition for treating cancer, comprising the E. coli strain of claim 7 as an active ingredient.
